(19) United States
(12) Patent Application Publication
(10) Pub. No.: US 2025/0082938 A1
(43) Pub. Date: Mar. 13, 2025

(54) SYSTEM AND METHOD FOR MODULATING NEURAL SYNCHRONY

(71) Applicant: Boston Scientific Neuromodulation Corporation, Valencia, CA (US)

(72) Inventor: Jianwen Gu, Richmond, TX (US)

(73) Assignee: Boston Scientific Neuromodulation Corporation, Valencia, CA (US)

(21) Appl. No.: 18/826,670

(22) Filed: Sep. 6, 2024

Related U.S. Application Data
(60) Provisional application No. 63/536,980, filed on Sep. 7, 2023.

(51) Int. Cl. A61N 1/36; A61N 1/05

(52) U.S. Cl. CPC A61N 1/36139; A61N 1/0534; A61N 1/36132; A61N 1/36171; A61N 1/36185

(57) ABSTRACT

Methods and systems for addressing issues related to synchrony in neural tissue signals. A spectrum of neural signals in a patient may be measured while the patient is asymptomatic, and compared to signals during a symptomatic episode to determine whether neural signals should be enhanced or disrupted. Depending on the change to neural signals, either enhancing or disrupting neural therapy is generated and confirmed or adjusted.

### CROSS-REFERENCE TO RELATED APPLICATIONS

This application claims the benefit of U.S. Provisional Patent Application Ser. No. 63/536,980, filed Sep. 7, 2023, which is incorporated herein by reference.

### BACKGROUND

Mane disease states affecting the nervous system are characterized by abnormal or unusual neural synchrony. Abnormally high neural synchrony can be associated with chronic pain, Parkinson's disease, epilepsy, and/or tremors. Abnormally low neural synchrony can be associated with Alzheimer's disease, schizoid disorders, and/or bipolar disorder. Normalizing synchrony has been shown to provide benefits, such as symptom control for Parkinson's disease, reduced Alzheimer's pathology, and/or to enhance cognition. Some research suggests that neural stimulation that is linked a patient's intrinsic neural oscillations may provide therapeutic benefits. New and alternative methods for configuring and delivering neurotherapy are desired.

### OVERVIEW

The present inventors have recognized, among other things, that a problem to be solved is the need for new and/or alternative systems and methods for modulating neural synchrony. Therapeutic benefits may be provided by reducing abnormally high neural synchrony for some conditions, and/or by enhancing abnormally low neural synchrony of other conditions.

A first illustrative and non-limiting example takes the form of a medical device system comprising: an implantable pulse generator having pulse generator circuitry including each of communication circuitry for communicating with one or more external devices, output circuitry for generating output pulses, and sensing circuitry for sensing one or more signals; a lead having a plurality of electrodes thereon and adapted for positioning of one or more electrodes in a patient and connectable to the implantable pulse generator; and an external device adapted to communicate with the implantable pulse generator, wherein the system is adapted for use in a patient having a neural condition with symptomatic episodes, characterized by: the implantable pulse generator being configured to receive an indication of a symptomatic episode; the implantable pulse generator being configured to record symptomatic neural activity during the symptomatic episode in response to the indication of a symptomatic episode; either the implantable pulse generator or the external device, using data transmitted from the implantable pulse generator, being adapted to perform the following: identifying characteristic frequencies of a non-symptomatic neural activity and characteristic frequencies of the symptomatic neural activity; and selecting a characteristic frequency differentiating the non-symptomatic neural activity from the symptomatic neural activity; and the implantable pulse generator, in response to the identifying and selecting, issuing neural stimulation to disrupt or enhance neural activity at the selected characteristic frequency.

Additionally or alternatively, the implantable pulse generator is configured to record the non-symptomatic neural activity from the patient while the patient is not experiencing a symptomatic episode.

Additionally or alternatively, the non-symptomatic neural activity is obtained from one of: a library of patients who have received an implanted lead; a patient sharing similar neural condition, age, gender, and/or disease state; or an average of other patients.

Additionally or alternatively, the implantable pulse generator or external device identifies characteristic frequencies by performing a mathematical transformation of data for the non-symptomatic neural activity and the symptomatic neural activity from a time domain to a frequency domain, and identifying peaks in the frequency domain for the non-symptomatic neural activity and the symptomatic neural activity.

Additionally or alternatively, the implantable pulse generator issuing neural stimulation to disrupt or enhance neural activity at the selected characteristic frequency is performed by the implantable pulse generator identifying a source of the symptomatic neural activity having the selected characteristic frequency.

Additionally or alternatively, the implantable pulse generator issuing neural stimulation to disrupt or enhance neural activity at the selected characteristic frequency is performed by the implantable pulse generator identifying a location of the symptomatic neural activity having the selected characteristic frequency and issuing neural stimulation to disrupt neural activity at the selected characteristic frequency by selecting electrodes for delivering the neural stimulation to deliver an electrical field to the location of the second neural activity.

Additionally or alternatively, the implantable pulse generator issuing neural stimulation to disrupt or enhance neural activity at the selected characteristic frequency is performed by the implantable pulse generator: a) sensing neural activity using each of a first subset of the plurality of electrodes and a second subset of the plurality of electrodes; b) determining which of the subsets of the plurality of electrodes used in a) attains a stronger signal at the selected characteristic frequency; and c) issuing the neural stimulation using the subset of the plurality of electrodes identified in b).

Additionally or alternatively, the implantable pulse generator issuing neural stimulation to disrupt or enhance neural activity at the selected characteristic frequency is performed by the implantable pulse generator: sensing further neural activity; filtering the sensed further neural activity to obtain signals at the selected characteristic frequency; determining a time of peaks at the characteristic frequency; and issuing the neural stimuli at the time of peaks to enhance the neural activity at the selected characteristic frequency.

Additionally or alternatively, the implantable pulse generator issuing neural stimulation to disrupt or enhance neural activity at the selected characteristic frequency is performed by the implantable pulse generator: sensing further neural activity; filtering the sensed further neural activity to obtain signals at the selected characteristic frequency; determining a time of peaks at the characteristic frequency; and issuing the neural stimuli at a delay relative to the time of peaks to disrupt the neural activity at the selected characteristic frequency.

Additionally or alternatively, the implantable pulse generator issuing neural stimulation to disrupt or enhance neural activity at the selected characteristic frequency is performed by the implantable pulse generator issuing pulses at a frequency having a period that is in the range of about 105% to 125% of a period of the characteristic frequency to disrupt the neural activity at the selected frequency.

Additionally or alternatively, the implantable pulse generator issuing neural stimulation to disrupt or enhance neural activity at the selected characteristic frequency is performed by the implantable pulse generator issuing pulses at the characteristic frequency to enhance the neural activity at the selected frequency.

Additionally or alternatively, the implantable pulse generator issuing neural stimulation to disrupt or enhance neural activity at the selected characteristic frequency is performed in response to a trigger, wherein the trigger is one of the following: the patient indicating occurrence of symptoms; a detection of the characteristic frequency occurring in further sensed neural activity; or an indication that the patient is or is about to engage in a cognitive or physical exercise.

Additionally or alternatively, the external device is a patient remote control or a clinician programmer. Additionally or alternatively, the lead has a proximal end for coupling to the pulse generator and a distal end carrying the plurality of electrodes, the distal end of the lead configured for positioning in the brain of the patient, and the pulse generator and lead together form an implantable system for deep brain stimulation.

Another illustrative and non-limiting example takes the form of a method of treating a patient having a neural condition with symptomatic episodes, the method comprising: recording first neural activity while the patient is not experiencing a symptomatic episode; receiving an indication of a symptomatic episode; recording second neural activity during the symptomatic episode; identifying characteristic frequencies of the first neural activity and characteristic frequencies of the second neural activity; selecting a characteristic frequency occurring only during the second neural activity; and issuing neural stimulation to disrupt neural activity at the selected characteristic frequency.

Additionally or alternatively, the step of identifying characteristic frequencies includes performing a mathematical transformation of data for first neural activity and the second neural activity from a time domain to a frequency domain, and identifying peaks in the frequency domain for the first neural activity and the second neural activity.

Additionally or alternatively, the step of issuing neural stimulation to disrupt the selected characteristic frequency includes identifying a source of the second neural activity having the selected characteristic frequency.

Additionally or alternatively, the method also includes identifying a location of the second neural activity having the selected characteristic frequency; and issuing neural stimulation to disrupt neural activity at the selected characteristic frequency by selecting electrodes for delivering the neural stimulation to deliver an electrical field at the location of the second neural activity.

Additionally or alternatively, the step of issuing neural stimulation is performed using an implanted neural stimulator having a lead with a plurality of electrodes, the neural stimulator configured to selectively use subsets of the plurality of electrode to sense neural activity, and the method further comprises: a) sensing neural activity using each of a first subset of the plurality of electrodes and a second subset of the plurality of electrodes; b) determining which of the subsets of the plurality of electrodes used in a) attains a stronger signal at the selected characteristic frequency; and c) issuing the neural stimulation using the subset of the plurality of electrodes identified in b).

Additionally or alternatively, the step of issuing neural stimulation comprises: sensing neural activity; filtering the sensed neural activity to obtain signals at the selected characteristic frequency; determining a time of peaks at the characteristic frequency; and issuing the neural stimuli at a delay relative to the time of peaks. Additionally or alternatively, the delay is selected to achieve a phase delay in the range of 120 to about 240 degrees.

Additionally or alternatively, the step of issuing neural stimulation is performed at a frequency having a period that is in the range of about 105% to 125% of a period of the characteristic frequency.

Additionally or alternatively, the steps of recording first neural activity and second neural activity are performed with an implanted system.

Additionally or alternatively, the step of issuing neural stimulation is performed in response to a trigger. Additionally or alternatively, the trigger is a patient input indicating occurrence of symptoms. Additionally or alternatively, the trigger is a detection of the characteristic frequency occurring only in the second neural activity.

Another illustrative and non-limiting example takes the form of a method of treating a patient having a neural condition with symptomatic episodes, the method comprising: obtaining first neural activity representing an expected neural activity spectral data; receiving an indication of a symptomatic episode; recording second neural activity during the symptomatic episode; identifying characteristic frequencies of the first neural activity and characteristic frequencies of the second neural activity; selecting a characteristic frequency occurring only during the second neural activity; and issuing neural stimulation to disrupt neural activity at the selected characteristic frequency.

Additionally or alternatively, the step of identifying characteristic frequencies includes performing a mathematical transformation of data for the second neural activity from a time domain to a frequency domain, and identifying peaks in the frequency domain for the second neural activity.

Additionally or alternatively, the step of issuing neural stimulation to disrupt the selected characteristic frequency includes identifying a source of the second neural activity having the selected characteristic frequency.

Additionally or alternatively, the method also includes identifying a location of the second neural activity having the selected characteristic frequency; and issuing neural stimulation to disrupt neural activity at the selected characteristic frequency by selecting electrodes for delivering the neural stimulation to deliver an electrical field at the location of the second neural activity.

Additionally or alternatively, the step of issuing neural stimulation is performed using an implanted neural stimulator having a lead with a plurality of electrodes, the neural stimulator configured to selectively use subsets of the plurality of electrode to sense neural activity, and the method further comprises: a) sensing neural activity using at least a first subset of the plurality of electrodes and a second subset of the plurality of electrodes; b) determining which of the subsets of the plurality of electrodes used in a) attains a stronger signal at the selected characteristic frequency; and c) issuing the neural stimulation using the subset of the plurality of electrodes identified in b).

Additionally or alternatively, the step of issuing neural stimulation comprises: sensing neural activity; filtering the sensed neural activity to obtain signals at the selected characteristic frequency; determining a time of peaks of the neural activity in the characteristic frequency; and issuing the neural stimuli at a delay relative to the time of peaks.

Additionally or alternatively, the delay is selected to achieve a phase delay in the range of 120 to about 240 degrees.

Additionally or alternatively, the step of issuing neural stimulation is performed at a frequency having a period that is in the range of about 105% to 125% of a period of the characteristic frequency.

Additionally or alternatively, the step of recording second neural activity is performed with an implanted system. Additionally or alternatively, the step of issuing neural stimulation is performed in response to a trigger. Additionally or alternatively, the trigger is a patient input indicating occurrence of symptoms. Additionally or alternatively, the trigger is a detection of the characteristic frequency occurring only in the second neural activity.

Another illustrative and non-limiting example takes the form of a method of treating a patient having a neural condition with symptomatic episodes, the method comprising: recording first neural activity while the patient is not experiencing a symptomatic episode; receiving an indication from a patient of a symptomatic episode; recording second neural activity during the symptomatic episode; identifying characteristic frequencies of the first neural activity and characteristic frequencies of the second neural activity; selecting a characteristic frequency occurring only in the first neural activity; and issuing neural stimulation to enhance neural activity at the selected characteristic frequency.

Additionally or alternatively, the step of identifying characteristic frequencies includes performing a mathematical transformation of data for the first neural activity and the second neural activity from a time domain to a frequency domain, and identifying peaks in the frequency domain for the first neural activity and the second neural activity.

Additionally or alternatively, the step of issuing neural stimulation to enhance the selected characteristic frequency includes identifying a source of neural activity having the selected characteristic frequency.

Additionally or alternatively, the method may include identifying a location of neural activity having the selected characteristic frequency; and issuing neural stimulation to enhance neural activity at the selected characteristic frequency by selecting electrodes for delivering the neural stimulation to deliver an electrical field at the identified location.

Additionally or alternatively, the step of issuing neural stimulation is performed using an implanted neural stimulator having a lead with a plurality of electrodes, the neural stimulator configured to selectively use subsets of the plurality of electrode to sense neural activity, and the method further comprises: a) sensing neural activity using at least a first subset of the plurality of electrodes and a second subset of the plurality of electrodes; b) determining which of the subsets of the plurality of electrodes used in a) attains a stronger signal at the selected characteristic frequency; and c) issuing the neural stimulation using the subset of the plurality of electrodes identified in b).

Additionally or alternatively, the step of issuing neural stimulation comprises: sensing neural activity; filtering the sensed neural activity to obtain signals at the selected characteristic frequency; determining a time of peaks of the neural activity in the characteristic frequency; and issuing the neural stimuli proximate to the time of peaks.

Additionally or alternatively, the step of issuing neural stimulation is performed at the characteristic frequency.

Additionally or alternatively, the step of issuing neural stimulation is performed in response to a trigger. Additionally or alternatively, the trigger is a patient input indicating occurrence of symptoms. Additionally or alternatively, the trigger is the absence of the characteristic frequency occurring only in the first neural activity.

Another illustrative and non-limiting example takes the form of a method of treating a patient having a neural condition with symptomatic episodes, the method comprising: obtaining first neural activity representing an expected neural activity spectral data; receiving an indication from a patient of a symptomatic episode; recording second neural activity during the symptomatic episode; identifying characteristic frequencies of the first neural activity and characteristic frequencies of the second neural activity;

selecting a characteristic frequency occurring only in the first neural activity; and issuing neural stimulation to enhance neural activity at the selected characteristic frequency.

Additionally or alternatively, the step of identifying characteristic frequencies includes performing a mathematical transformation of data for the second neural activity from a time domain to a frequency domain, and identifying peaks in the frequency domain for the second neural activity.

Additionally or alternatively, the step of issuing neural stimulation to enhance the selected characteristic frequency includes identifying a source of neural activity having the selected characteristic frequency.

Additionally or alternatively, the method also includes identifying a location of the second neural activity having the selected characteristic frequency; and issuing neural stimulation to enhance neural activity at the selected characteristic frequency by selecting electrodes for delivering the neural stimulation to deliver an electrical field at the location of the second neural activity.

Additionally or alternatively, the step of issuing neural stimulation is performed using an implanted neural stimulator having a lead with a plurality of electrodes, the neural stimulator configured to selectively use subsets of the plurality of electrode to sense neural activity, and the method further comprises: a) sensing neural activity using at least a first subset of the plurality of electrodes and a second subset of the plurality of electrodes; b) determining which of the subsets of the plurality of electrodes used in a) attains a stronger signal at the selected characteristic frequency; and c) issuing the neural stimulation using the subset of the plurality of electrodes identified in b).

Additionally or alternatively, the step of issuing neural stimulation comprises: sensing neural activity; filtering the sensed neural activity to obtain signals at the selected characteristic frequency; determining a time of peaks of the neural activity in the characteristic frequency; and issuing the neural stimuli proximate to the time of peaks.

Additionally or alternatively, the step of issuing neural stimulation is performed at the characteristic frequency.

Additionally or alternatively, the step of issuing neural stimulation is performed in response to a trigger. Additionally or alternatively, the trigger is a patient input indicating occurrence of symptoms. Additionally or alternatively, the trigger is the absence of the characteristic frequency occurring only in the first neural activity.

The preceding method examples may be performed as well by system for treating neurological disorders, such as a deep brain stimulation (DBS) system including an implantable pulse generator, an associated lead, and one or more external devices.

This overview is intended to provide an introduction to the subject matter of the present patent application. It is not intended to provide an exclusive or exhaustive explanation. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example deep brain stimulation (DBS) system as implanted;

FIG. 2 illustrates details of a directional DBS lead;

FIGS. 3A-3C show illustrative methods in block form;

FIG. 4 illustrates a frequency domain comparison; and

FIGS. 5-6 show illustrative user interfaces.

### DETAILED DESCRIPTION

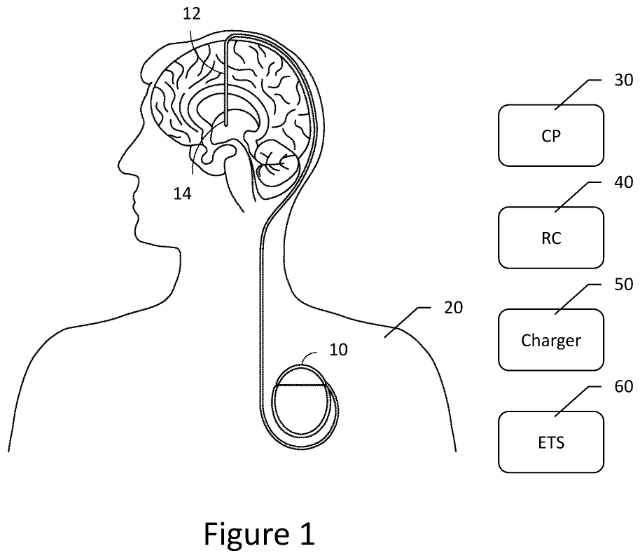

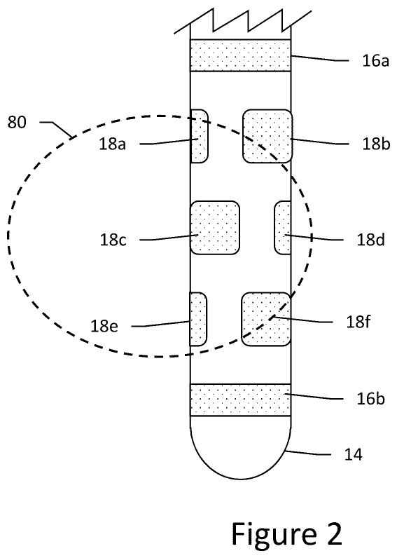

FIG. 1 shows an illustrative deep brain stimulation (DBS) system implanted in a patient. The system comprises a pulse generator 10, shown implanted in the pectoral region of a patient 20. The pulse generator 10 is coupled to a lead 12 which extends subcutaneously to the head of the patient 20, through a burr hole formed in the patient's skull, and then into the brain of the patient. In the example shown, the lead 12 includes a plurality of electrodes positioned near the distal end 14 of the lead, such as shown below in FIG. 2. The lead 12 may be placed at any suitable location of the brain where a target for therapy is identified. For example, a lead 12 may be positioned so that the distal end 14 is near the mid-brain and/or various structures therein that are known in the art for use in providing stimulation to treat various diseases. The electrodes on the lead 12 may be used for issuing stimulus pulses/signals, and/or for sensing signals in the brain.

DBS may be targeted, for example, and without limitation, at neuronal tissue in the thalamus, the globus pallidus, the subthalamic nucleus, the pedunculopontine nucleus, substantia nigra pars reticulata, the cortex, the globus pallidus externus, the medial forebrain bundle, the periaquaductal gray, the periventricular gray, the habenula, the subgenual cingulate, the ventral intermediate nucleus, the anterior nucleus, other nuclei of the thalamus, the zona incerta, the ventral capsule, the ventral striatum, the nucleus accumbens, and/or white matter tracts connecting these and other structures. Data related to DBS may include the identification of neural tissue regions determined analytically to relate to side effects or benefits observed in practice. “Targets” as used in the art may be brain structures associated with therapeutic benefits, and avoidance regions or “Avoid” regions may be brain structures associated with side effects.

Conditions to be treated may include dementia, Alzheimer's disease, Parkinson's disease, various tremors, depression, anxiety or other mood disorders, sleep related conditions, etc. Therapeutic benefits may include, for example, and without limitation, improved cognition, alertness, and/or memory, enhanced mood or sleep, avoidance of pain or tremor, reduction in motor impairments, and/or preservation of existing function and/or cellular structures, such as preventing loss of tissue and/or cell death. Therapeutic benefits may be monitored using, for example, patient surveys, performance tests, and/or physical monitoring such as monitoring gait, tremor, etc. Side effects can include a wide range of issues such as, for example, and without limitation, reduced cognition, alertness, and/or memory, degraded sleep, depression, anxiety, unexplained weight gain/loss, tinnitus, pain, tremor, etc. These are just examples, and the discussion of ailments, benefits and side effects is merely illustrative and not exhaustive.

The illustrative system of claim 1 includes various external devices. A clinician programmer (CP) 30 may be used to determine/select therapy programs, including steering (further explained below) as well as stimulation parameters. Stimulation parameters may include amplitude of stimulation pulses, frequency or repetition rate of stimulation pulses, pulse width of stimulation pulses, and more complex parameters such as burst definition, as are known in the art. Biphasic square waves are commonly used, though nothing in the present invention is limited to biphasic square waves, and ramped, triangular, sinusoidal, monophasic and other stimulation types may be used as desired. The CP 30 may be, for example, a laptop or tablet computer and can be used by a physician, or at the direction of a physician, to obtain data from and provide instructions to the pulse generator 10 via suitable communications protocols such as Bluetooth or MedRadio or other wireless communications protocols, and/or via other modalities such as inductive telemetry.

A patient remote control (RC) 40 can be used by the patient to perform various actions relative to the pulse generator 10. These may be physician defined options, and may include, for example, turning therapy on and/or off, entering requested information (such as answer questions about activities and therapy benefits and side effects), and making (limited) adjustments to therapy such as selecting from available therapy programs and adjusting, for example, amplitude settings. The RC 40 can communicate via similar telemetry as the CP 30 to control and/or obtain data from the pulse generator 10. The patient RC 40 may also be programmable on its own, or may communicate or be linked with the CP 30.

A charger 50 may be provided to the patient to allow the patient to recharge the pulse generator 10, if the pulse generator 10 is rechargeable. Some pulse generators 10 are not rechargeable, and so the charger 50 may be omitted. The charger 50 can operate, for example, by generating a varying magnetic field to activate an inductor associated with the pulse generator 10 to provide power to recharge the pulse generator, using known methods.

Some systems may include an external test stimulator (ETS) 60. The ETS 60 can be used intraoperatively to test therapy programs after the lead 12 has been positioned in the patient to determine whether therapy is or can work for the patient 20. For example, an initial implantation of the lead 12 can take place using, for example, a stereotactic guidance system, with the pulse generator 10 temporarily left out. The lead 12 may have a proximal end thereof connected to an intermediate connector (sometimes called an operating room cable) that couples to the ETS 60. After lead 12 has been implanted and coupled to the ETS 60, the ETS can be programmed using the CP 30 with various therapy programs and stimulation parameters, which are tested to determine therapy effects. Lead position may be adjusted, as needed during this process. Once therapy suitability for the patient is established, the permanent pulse generator 10 is implanted and the lead 12 is connected thereto, with the ETS then being removed from use.

The pulse generator 10 may include operational circuitry for generating output stimulation programs and/or pulses in accordance with stored instructions, as well as for sensing signals including electrical signals emanating from various tissues. Some examples of prior versions of such circuitry, as well as planned future examples, may be found in U.S. Pat. No. 10,716,932, the disclosure of which is incorporated herein by reference. Pulse generator circuitry may include that of the various commercially known implantable pulse generators for spinal cord stimulation, Vagus nerve stimulation, and deep brain stimulation as are also well known. Additional examples of the pulse generator 10, CP 30, RC 40, Charger 50, and ETS 60 can be found, for example and without limitation, in U.S. Pat. Nos. 6,895,280, 6,181,969, 6,516,227, 6,609,029, 6,609,032, 6,741,892, 7,949,395, 7,244,150, 7,672,734, 7,761,165, 7,974,706, 8,175,710, 8,224,450, and 8,364,278, the disclosures of which are incorporated herein by reference in their entireties.

The pulse generator may include, for example and without limitation, a power supply in the form of one or more batteries, which may be rechargeable as noted above, or may be primary cells (not rechargeable). Pulse generator circuitry may include a current or voltage generating architecture, having a plurality of channels/outputs of current and or voltage. For example, a collection of current mirrors each providing a discrete current output (as source or sink) which may be summed together via a low impedance switching network may serve as the output architecture for a current controlled system. Pulse generator circuitry may include an input signal selector (such as a multiplexor or switch array) that allows one or more selected electrodes to serve as anodes/cathodes for a sensing electrode pair to obtain desired signals, such as signals emanating from portions of the brain. The received signal may be filtered in the analog domain to remove non-physiologic signals (DC, line noise, and/or high frequency for example), amplified, and digitized for analysis by a microcontroller also present in the pulse generator circuitry. Digital filtering may also be performed, as needed. The received signals may be stored for later communication to an external device using wireless technology, and noted above.

FIG. 2 illustrates details of a directional DBS lead. The distal end 14 is shown, and a plurality of electrodes are shown as well. Two ring electrodes 16a, 16b (collectively ring electrodes 16) can be provided as shown, and a number of segmented electrodes are shown at 18a, 18b, 18c, 18d, 18e, 18f (collectively, segmented electrodes 18). Each electrode 16, 18 is separately addressable in the system, such as by using a pulse generator having multiple independent current control (MICC), or multiple voltage sources. MICC is a stimulus control system that provides a plurality of independently generated output currents that may each have an independent quantity of current. The use of MICC can allow spatially selective fields to be generated during therapy outputs. The term “fractionalization” may refer to how the total current issued by the pulse generator via the electrodes is divided up amongst the electrodes 16, 18 on the lead.

It should be noted that the pulse generator canister may serve as an indifferent electrode or as a return electrode for therapy outputs; if desired, one of the electrodes (such as a ring electrode 16 or one or more of the segmented electrodes 18) may instead be used as a return electrode. Thus, for example, the electrodes on the lead may serve as cathodes while pulse generator canister serves as an anode during one phase of stimulation pulse delivery. In another example, some of the lead electrodes 16, 18 serve as cathodes, while other lead electrodes serve as anodes during one phase of stimulation pulse delivery. Any suitable combination and quantity of anodes and cathodes may be used for therapy purposes, and any lead electrode and/or the housing electrode can be used in any of these roles, as needed.

Examples of electrical leads with segmented or directional lead structures are shown, for example and without limitation, in US PG Pat. Pubs. 20100268298, 20110005069, 20110078900, 20110130803, 20110130816, 20110130817, 20110130818, 20110238129, 20110313500, 20120016378, 20120046710, 20120071949, 20120165911, 20120197375, 20120203316, 20120203320, 20120203321, 20130197602, 20130261684, 20130325091, 20130317587, 20140039587, 20140353001, 20140358207, 20140358209, 20140358210, 20150018915, 20150021817, 20150045864, 20150021817, 20150066120, 20130197424, and 20150151113, and U.S. Pat. Nos. 8,483,237 and 8,321,025, the disclosures of which are incorporated herein by reference.

MICC used with a directional lead can facilitate precise therapy targeting. For example, a directional lead as shown in FIG. 2 may be used to generate a stimulation field as illustrated at 80 in FIG. 2. The outer boundary of field 80 may be understood as representing an equipotential or equal field boundary, within which the electrical field is higher than an activation threshold, and outside of which the electrical field is below the threshold, for purposes of illustration. An activation threshold may represent or approximate a voltage/field threshold at which neural cells will activate or “fire”. Activation thresholds may be determined on a population basis, such as by relating to a voltage/field at which a 50% likelihood of activation of 50% of the cell population is determined, thought other boundaries/thresholds can be used. The shape of the field can be adjusted, as described variously in the references incorporated by reference above, by modifying the fractionalization of current issued via the electrodes using MICC. An output creating an activation field boundary as shown at 80 may be (roughly) generated by using electrode 18c as a cathode, and surrounding electrodes 18a, 18e, and 18d as anodes, for example. The actual characteristics of fractionalization may be more sophisticated than this simple example.

The boundary shown at 80 in FIG. 2 can be used to illustrate stimulation field effects, and can be generated for purposes of display using stimulation field modeling (SFM). In SFM, the tissue is modeled, for example, using finite element models in which the lead body is treated as an insulator, surrounded by a thin encapsulation sheath, and surrounded by neural tissue. The neural tissue may be modeled as isotropic and homogenous, though more sophisticated modelling can also be used if desired. A set of model voxels are defined around the lead, breaking up the volume into small segments, each of which can be analyzed within the model. The outer boundaries of the SFM can be determined using a population-based activation threshold as described above. The result can be that at a given fractionalization and total stimulation current, an SFM can be generated as a three-dimensional surface surrounding a portion of the lead and encompassing a volume of neural tissue. Field 80 may be, for example, understood as a two-dimensional representation of a slice of the SFM. As noted, the SFM can be used as a visual tool for illustrating, to a patient or physician, what tissue is or is not being stimulated by a given fractionalization and total current.

Because the electrodes 16, 18 are separately addressable from one another, each electrode 16, 18 may also be used as a separate sensing node. The operational circuitry of the implantable system may include, for example and without limitation, analog and digital filtering circuitry, as well as an amplifier, allowing the operational circuitry to selectively sense one or more signals in the patient. For example, a switching circuit may be provided to allow sensing to be performed using a desired pair or other combination of electrodes 16, 18 (as well as the electrode of the implantable pulse generator housing, as desired).

Multiple sensing channels may be available, if desired. The incoming signal can be DC filtered and subjected to any other desired analog filtering before going to an amplifier which amplifies the received signal (sometimes using variable gain) to an amplitude that can be converted to a digital data stream.

A frequency selective digital filter may be used to, for example, remove residual non-physiological signals (such as having a 50 Hz or 60 Hz filter to remove line noise in the environment), as well as to obtain a frequency band of specific interest. For example, digital filtering may be useful to identify some of the signals present in the brain, such as:
Delta Waves, found in the range of about 0.5 to 3.5 Hz. Excess delta frequencies can be associated with sleep disturbance, anxiety and difficulty focusing.
Theta Waves, found in the range of about 3.5-7 Hz. Sometimes associated with movement and/or waking from sleep; less studied in humans.
Alpha Waves, found in the range of about 8-13 Hz. Increased alpha has been hypothesized to improve cognition and/or reduce anxiety or depression.
Beta Waves, found in the range of about 18-25 Hz. Increased beta may relate to the ability to focus and other cognitive skill.
Gamma Waves, found in the range of about 30-60 Hz. Reduced gamma can negatively affect mood and memory; increased gamma may do the opposite.
   In some examples, filtering may be performed to obtain a signal of about 60 Hz or less, or 50 Hz or less (depending on the location and whether 50 Hz or 60 Hz line frequency is used), and the obtained signal may be recorded for later processing as needed. For example, in some examples that follow, a trigger may prompt a pulse generator to record certain signals during a symptomatic episode of neural activity believed to be abnormal. A spectrum of signals in the range of about 0.5 to about 60 Hz may be of interest, and so may be recorded in the pulse generator at, for example, a sampling frequency in the range of more than 200 Hz, including, for example, 256 Hz, 512 Hz, and/or 1 kHz, or higher. Such signal data can be recorded in the pulse generator memory (or ETS memory) and transmitted to an external device, such as the patient RC 40 and/or a CP 30 (referring now to FIG. 1), for further processing.

In implementation, the processing power of a CP 30 (which may be a tablet computer, laptop computer, etc.) or even an RC 40 (which may be a smartphone, for example) should be sufficient to perform the analysis of frequency spectrum, which may include a Fourier Transformation. Other data processing to identify frequency components of interest may include, for example and without limitation, principle components analysis, wavelet decomposition, and/or any orthogonal or non-orthogonal basis function decomposition. Such processing may strain the resources of a current generation pulse generator 10 or ETS 60, require excess battery/power consumption, or take a relatively long period of time, though future iterations of these technologies may be more suited to these activities. In still other examples, spectrum data may be transmitted to a central server from the CP 30 and/or RC 40. Some examples may use a bedside monitor that may also obtain periodic data from a pulse generator such as by daily or weekly download, if a bedside or other home monitoring system is provided (not shown in FIG. 1, but well known in the art).

Other processes, such as digitally filtering a signal to a narrow passband with high quality factor, may be more readily performed in pulse generator. That is, for example and without limitation, in some of the following examples, the pulse generator may be tasked with determining signal strength at a particular frequency or narrow frequency band. Doing so may be relatively easier, as the filtering can be performed in the digital domain by supplying well-crafted filtering coefficients to a digital filter or even, if desired, a dedicated digital signal processing chip.

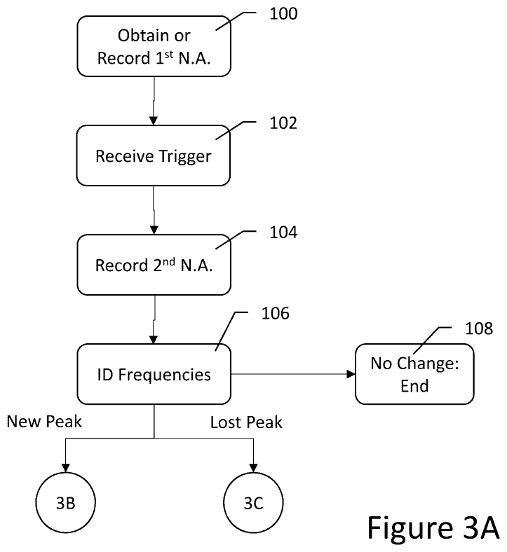

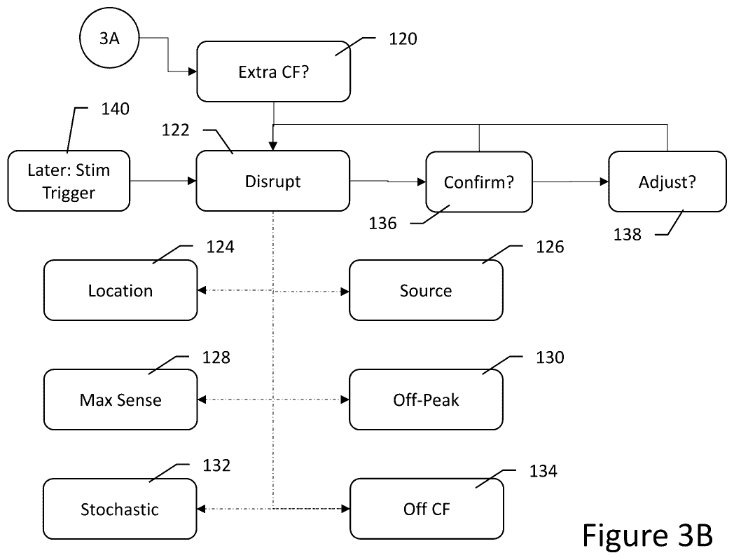

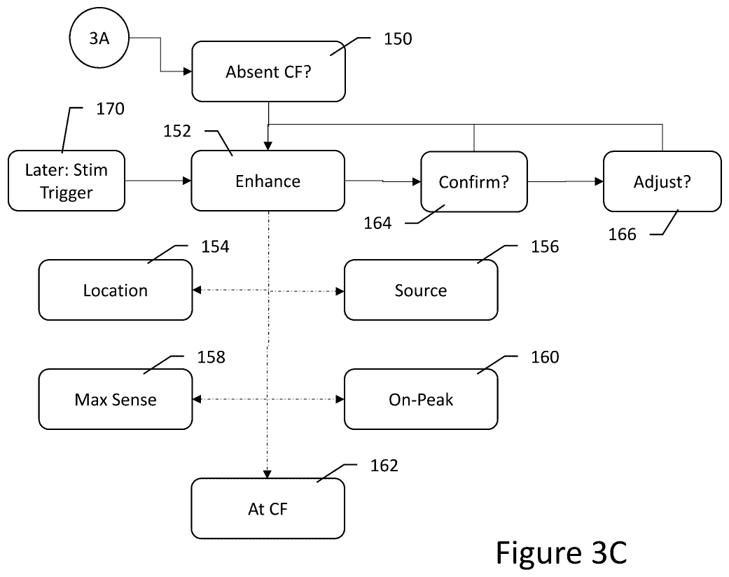

FIG. 3A shows an illustrative method. A first neural activity (N.A.) is obtained or recorded at 100. Obtaining a first N.A. may be performed, for example, using data from other patients. For example, the idea in some examples may be to obtain data for a patient not currently experiencing abnormal neural activity. Some patients may experience chronic symptoms, such that a recording of first neural activity while the patient is not experiencing symptoms would be unavailable. For such patients, “healthy” normative data may be used instead. For example, neural activity data during asymptomatic periods may be recorded from another person having similar characteristics (such as comparable age, neural condition, gender, brain anatomy and/or other characteristics) and used as the first neural activity. Likewise, an average of similar patient data can be obtained. In another example, neural activity may be obtained across a population of patients and characteristic frequencies for non-symptomatic states can be obtained from the patient population data. In another example, data may be obtained from other patients with a similar or related neurological disease or symptomatic episodes. Data may be obtained from such “similar” patients who do have periods of asymptomatic conditions but who also have a lead positioned similar to that of the patient being treated, as the lead position may affect the set of characteristic frequencies that are obtained. Data may be, for example, obtained from a different patient that has been successfully treated using a DBS system, for example. Thus, rather than a patient-specific comparison, a set of characteristic frequencies from normative data may be obtained at block 100.

Recording a first N.A. at block 100 may include, for example, recording several seconds of received neural activity data using one or more channels. For example, a plurality of channel specific recordings may be made. Referring to FIG. 2 again briefly, a selected subset of available sensing combinations may be chosen for recording “normal” N.A., and each combination of sensing electrodes defines a “sensing vector” therebetween. In an example, plural and spatially diverse sensing vectors for a single lead may be obtained such as by choosing a relatively more proximal electrode (ring 16a or one of segmented electrodes 18a, 18b) for use with a distant indifferent electrode (such as the pulse generator canister) as one sensing vector, a more distal electrode (ring 16b or one of segmented electrodes 18e, 18f) with the indifferent electrode as a second sensing vector, and then two of the lead electrodes as opposing poles for a third sensing vector, the idea being to obtain spatially diverse sensing data. This is just an example. In other examples, a single sensing vector can be defined.

If more than one lead is implanted in a patient, sensing vectors may be defined using one sensing electrode on a first lead, and a second electrode on a second lead; again, plural vectors can be configured. It may be desirable to record two or more sensing vectors at the same time, to allow simultaneous monitoring. In some examples, two or more sensing vectors may be recorded at non-overlapping times. The data from block 100 may be recorded in a periodic manner, if desired, such as on a daily or weekly basis, as desired.

A trigger is then received at 102. A trigger may be an input that indicates to the implantable pulse generator (or an ETS, if desired) that the patient is experiencing symptoms of a neural condition. For example, an implantable system may include an accelerometer configured to determine the patient is experiencing uneven gait, or a tremor, and when such a condition arises, this may indicate a symptomatic episode is occurring, and can be identified as a trigger in block 102. In another example, a patient may use a patient RC to indicate that symptoms are occurring, again serving as a trigger of a symptomatic episode. For example, a patient may indicate the occurrence of a seizure, a manic episode, a depressive episode, or a hallucination.

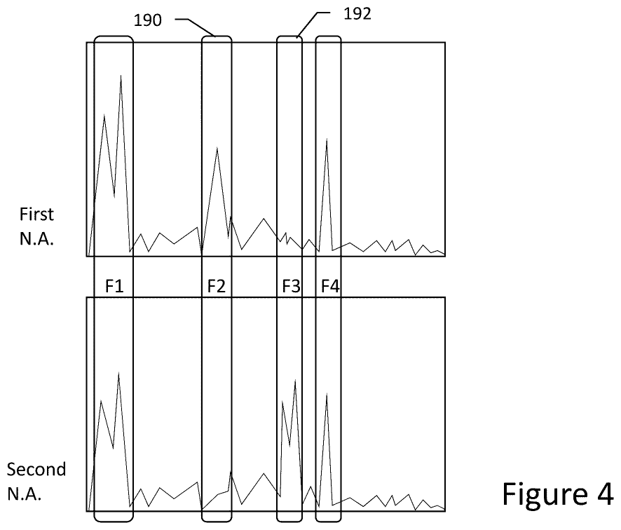

In response to the trigger 102, the system records a second N.A. at 104. The data recorded at 104 may be the same as that recorded at 100, if desired and in some examples, though there can be variation between block 100 and block 104. Characteristic frequencies are then identified at 106. Block 106 may include, for example and without limitation, the implantable device, or an external (RC, CP, bedside monitor, or a computer coupled to remote database server), analyzing the data gathered at 100 and 104 to identify peak frequency data. For example, a fast Fourier transformation (FFT) may be executed, converting the time domain sensed data to the frequency domain. Peaks frequency data may be identified as characteristic frequencies, as explained relative to FIG. 4.

Referring to FIG. 4, a comparison of hypothetical frequency bands of sensed data is shown. A First N.A. is shown in the upper graphic, and a Second N.A. is shown in the lower graphic, as indicated. As described above, the Second N.A. may correspond to a patient experiencing a symptomatic episode a neural phenomenon. The graphs may show, for example, FFT on a block of sensed data spanning a frequency range from about 0.5 Hz to about 60 Hz. While FFT is shown in FIG. 4, any mathematical transformation that allows conversion of sensed time domain data to allow frequency analysis to take place may be used. FFT is one such analysis. Others may include principle component analysis, wavelet decomposition, or any other orthogonal or non-orthogonal basis function decomposition, each of which may be understood as converting time domain data to the frequency domain, as frequencies can be analyzed after the conversion or decomposition.

As can be seen, there are several frequency peaks across the band of frequencies shown, including at F1, F2, F3 and F4. Characteristic frequencies may include such peaks; determining which peaks to identify as characteristic peaks may be performed in any suitable manner. In some examples, a peak that is at least twice the average amplitude across the transformation, or which exceeds twice the root-mean-squared (RMS) amplitude of the signal may be considered. In other examples, statistical analysis may be used to identify peaks that exceed the average signal by two, three, or more standard deviations or variances.

Comparing the First N.A. to the Second N.A., F1 and F4 remain generally consistent, however, frequency F2, at 190, drops significantly when the symptomatic episode is ongoing, and frequency F3, at 192, is significantly stronger. Any suitable measure of “difference” may be used; in an example, the overall spectrum strength is observed, and any change at a given frequency or band of frequencies that exceeds 25% or some other fraction thereof can be identified as a change or difference from the First N.A. to the Second N.A. The number (1, 2, or more) and type (increase, decrease, etc.) of changes can vary with patient and symptom, and the illustrative example is not intended to precisely mimic actual signals. For some patients, a single change will be observed; the example in FIG. 4 is provided to illustrate an increase at a characteristic frequency at F3, and a decrease at a characteristic frequency at F4. The phrase “at a frequency” should be understood as meaning across a narrow band of frequencies, such as +/−1-5, or +/−1-3 Hz about a center frequency, or +/−1% to 5% of the center frequency, for example and without intending a specific limitation on the concept.

Returning to FIG. 3A, block 106 indicates that the characteristic frequencies are identified. Of particular interest are any frequencies that change from the 1st N.A. recorded at 100 to the 2nd N.A. recorded at 104. If there is no change in the characteristic frequencies, the method may terminate as indicated at 108. If one or more new peaks appear in the spectral analysis, the method continues at FIG. 3B. If one or more peaks are lost or no longer appear, the method continues at FIG. 3C.

In an alternative example, blocks 100 and 104 may reflect an in-clinic or in-hospital sequence of events. If in-clinic or in-hospital, the data gathered in each step may be data from an electro-encephalogram (EEG) machine, in which case electrodes will have been positioned on the skull of the patient, or may be inserted/implanted/etc. as desired. The frequency identification at 106 can then be performed by the EEG machine or by a computer coupled to or receiving data from the EEG machine. It may still be useful to have the implanted system record data as well, at least to allow configuration of the sensing circuits of the implanted system to ensure that a best sensing vector, for example, can be used to identify a signal/frequency of interest.

Turning to FIG. 3B, with an extra characteristic frequency (“CF” in the figures) arising, as indicated at 120, the next step in the method is to disrupt that signal, as indicated at 122. Several approaches to disruption 122, which may be used individually or together, are available.

The location at which a signal generates may be identified at 124, and therapy generated to target the source of the signal. For example, multiple sense vectors may be available in the system. By monitoring for the particular frequency of the extra characteristic frequency signal, and comparing relative strength across multiple sense vectors, the location where the signal originates from may be estimated. Monitoring for a particular frequency can be performed using bandpass filtering, for example, or by the use of the mathematical transformations described previously.

Then, using, for example, a directional lead as in FIG. 2, the output currents or voltages of the system may be manipulated to issue a modulating signal to the source. The modulating signal may be delivered at any frequency, bandwidth, and amplitude (and waveform shape) suitable to the system, within system limitations. In another example, the signal source location may be identified using externally applied imaging and/or measurement systems, such as an EEG, a functional MRI, etc. Again, therapy may be targeted to the location. While much of the present focus is on the use of electrical neuromodulation, other therapy approaches such as photo-biomodulation may be used, or alternative, such as ablation, may be used, as desired, to target a location 124, if desired.

A source of the signal may be identified, as indicated at 126, and modulation directed to the particular source. For example, using a database of patients having similar disease states, symptoms, and, in particular, frequency bands or at least a similar additional frequency present, the source may be identified by reference to the patient data in the database. This step may be somewhat similar to the use of location 124, however, source targeting may include frequency selection or use of other factor, such as identifying a neural structure that can affect a source which is within a therapy range. For example, if a nerve fiber can be targeted for stimulation/modulation, where the nerve fiber is connected to a neural structure that can be identified as the source of a characteristic frequency (again, possibly with the aid of external imaging or measurement systems), the fiber may be subjected to stimulation/modulation signals to attempt to limit the effect of the source of the new characteristic frequency. Other therapy types, such as ablation, may be used as well or instead, once the analysis has identified the frequency of interest and a signal source.

Maximum amplitude 128 is another variant on the use of location 124. Here, the system may take an analytical approach to sensing first. That is, neural activity (during symptomatic episodes) can be monitored using at least first and second sensing electrode pairs, or other subsets (combinations of three or more electrodes may be used, if desired, for sensing, though this may prove less sensitive to location in some examples) of the plurality of electrodes available in a system. Using frequency filtering, the amplitude within a narrow band of frequencies about the extra characteristic frequencies may be monitored to determine which sensing electrode combination obtains a stronger or maximal signal at the additional characteristic frequency. Subsequently neural stimulation signals can be issued using those same electrodes as output electrodes, placing therapy in the spatial location of highest signal amplitude.

Additional approaches may use an understanding of interfering waves/frequency characteristics. For example, output therapy pulses may be delivered at the frequency of the extra characteristic frequency identified by the system, however, those pulses may be delivered at an offset relative to the signal peaks—that is, off-peak 130. This can create destructive interference with the signal. For example, therapy delivery out of phase, that is off-peak by at least 90° of the phase, and more preferably in the range of about 120° to about 240°, or about 135° to about 225°, or about 150° to about 210°, or about 170° to 190°, or about 180°, out of phase relative to the intrinsic signal. Even if the output signal is a square wave, and the intrinsic signal is not, the output being out of phase should have an interference effect, reducing the propagation of the signal. In another alternative, a sinusoidal or other shaped signal may be used to deliver a signal that is more correctly characterized as being out of phase, rather than off-peak 130. In some examples, filtering may be used to limit the signal frequencies that are being observed when attempting to identify a peak. The filtering itself can cause the analyzed signal to undergo a phase shift; before issuing a signal that is off-peak (as noted at 130 in FIG. 3B) and/or on-peak (as noted at 160 in FIG. 3C), the phase shift caused by the filtering may be addressed by adjusting the timing of therapy outputs to account for filtering-induced phase shift. That is, therapy may be output at a point in time that is adjusted relative to the observed peak in the filtered signal to account for phase shift. Such an output may be generated by first assuming a cyclic nature of the sensed signal, so that a peak in the sensed signal is identified and the output generated after the peak is identified using a time delay determined as a fraction (1% to 150%) of the period of the signal, plus or minus filtering-induced phase changes. In other examples, the output signal can be generated at a rate that is chosen to match the characteristic frequency being enhanced or interfered with, and the timing of the output signal may be adjusted repeatedly until a desired effect (disrupt or enhance) occurs.

In another example, a stochastic approach can be taken to introduce signal noise in the range of the additional characteristic frequency, as indicated at 132. For example, outputs can be generated with a frequency that will vary above and below the characteristic frequency. This may be performed using square waves having a varying inter-pulse period that varies above and below the period of the additional characteristic frequency. If sinusoidal waves, or approximations thereof, are available, the frequency may be varied above and below a center point that is at or near the additional characteristic frequency. Stochastic noise 132 stimulation or modulation signals may be selected for use, for example, in response to particular patient symptoms, rather than in response to a particular signal frequency, such as by issuing stochastic signaling directed to a location associated with an epileptic seizure.

In another example, therapy can be generated using a frequency which is near, but off of the additional characteristic frequency, as indicated at 134. For example, an overdrive frequency would be above the characteristic frequency, such as in the range of about 105% to about 150% of the characteristic frequency, or about 105% to about 125% of the additional characteristic frequency. A lower frequency can be used instead, such as in the range of about 75% to about 99% of the additional characteristic frequency. These various approaches 124, 126, 128, 130, 132, 134 may be used in combinations, as desired.

The disruption signal generated at 122 may be issued and the patient response monitored. Therapy may be preliminarily confirmed by monitoring for a change in the additional characteristic frequency signal, as indicated at 136. Whether a change in the additional characteristic frequency signal translates to an effect on patient symptoms may be determined as well, for example, using monitoring of any signals that were relied upon to identify the symptoms in the first place, as taught above relative to block 102 in FIG. 3A. If the characteristic frequency and/or symptoms are not affected, further adjustments may be made, as indicated at 138. Adjustment 138 may include switching from one disruption strategy to another, for example, or combining two such strategies, or changing a parameter of a strategy in use, such as increasing or decreasing one or more of amplitude, frequency, etc.

In some examples, the result may be considered a disruption program that can be stored for later execution in the device. That is, if a change is observed, and the additional frequency appears to be disrupted, the therapy program can be stored to memory, and later called again in response to a trigger. To do so, the method may include block 140, which would trigger use of the disruption signal by entering the method at block 122, as shown. A trigger at block 140, prompting use of the stored disrupting signal, may be the same as at block 102 in FIG. 3A. In another example, the trigger at 140 may rely on sensing for the additional characteristic frequency; if the additional characteristic frequency is observed, that may be taken as indicating occurrence, likelihood, or possible future onset, of symptoms, and can be used to trigger the disrupting therapy. Block 140 may also include storing one or more sensed signals preceding the initiation of therapy—for example, if sensing data is stored in a looping register (such as a first-in-first-out memory), the signals associated with or preceding the trigger can be stored for later physician review, if desired. Moreover, blocks 122 and/or 136 may also include recording one or more neural signals, to observe for example what occurs while the disruption therapy is delivered at 122, or to observe neural activity after therapy delivery.

Returning to FIG. 3A, if a frequency peak is lost at block 106, the method may proceed to FIG. 3C. Here, a characteristic frequency is absent, as indicated at 150. The next step is to deliver a therapy output that enhances neural activity at the absent frequency, as indicated at 152. This may be done several ways.

The location at which a missing signal was generated may be identified at 154, and therapy generated to target the source of the signal. For example, multiple sense vectors may be available in the system. By analyzing the previously recorded First N.A., monitoring for the particular frequency of the extra characteristic frequency signal, and comparing relative strength across multiple sense vectors, the location where the signal originated from may be estimated. Then, using, for example, a directional lead as in FIG. 2, the output currents or voltages of the system may be manipulated to issue a modulating or stimulating signal to the source. The modulating or stimulating signal may be delivered at any frequency, bandwidth, and amplitude (and waveform shape) suitable to the system, within system limitations. In another example, the signal source location 154 may be identified using externally applied imaging and/or measurement systems, such as an EEG, a functional MRI, etc. Again, therapy may be targeted to the location.

Again, using a pre-recorded, First N.A., a source of the signal may be identified, as indicated at 156, and modulation directed to the particular source. For example, using a database of patients having similar disease states, symptoms, and, in particular, a “normal” frequency of neural activity that disappears or becomes attenuated during a symptomatic episode, the source may be identified by reference to the patient data in the database. This step may be somewhat similar to the use of location 154, however, source targeting may include frequency selection or use of other factor, such as identifying a neural structure that can affect a source which is within a therapy range. For example, if a nerve fiber can be targeted for stimulation/modulation, where the nerve fiber is connected to a neural structure that can be identified as the source of a characteristic frequency (again, possibly with the aid of external imaging or measurement systems), the fiber may be subjected to stimulation/modulation signals to attempt to stimulate the neural structure to again generate the characteristic frequency that went missing or was attenuated during the symptomatic episode.

Maximum amplitude 158 is another variant on the use of location 124. Here, the system may take an analytical approach to sensing first. That is, neural activity (during times lacking any symptoms) can be monitored using at least first and second sensing electrode pairs, or other subsets (combinations of three or more electrodes may be used, if desired, for sensing, though this may prove less sensitive to location in some examples) of the plurality of electrodes available in a system. Using frequency filtering, the amplitude within a narrow band of frequencies about the missing characteristic frequencies may be monitored to determine which sensing electrode combination obtains a stronger or maximal signal at the missing characteristic frequency. Subsequently neural stimulation signals can be issued using those same electrodes as output electrodes, placing therapy in the spatial location of highest signal amplitude.

In some examples, blocks 154, 156 and 158 may be performed after first making the observations of FIG. 3A, then waiting for cessation of symptoms before performing additional sensing to identify any of location 154, source 156, or maximum sensing position 158. Then, later, the routine of delivering therapy to enhance the missing characteristic signal at block 152, following with confirmation 164 and adjustment 166 as explained further below, may all take place when symptoms again arise.

In some examples the missing characteristic frequency may still be present, but merely attenuated to no longer be dominant. If so, another approach may be to issue pulses at or near the time of signal peaks, as indicated at 160. For example, if the attenuated characteristic frequency can be sensed using, for example, narrow bandpass filtering, output pulses may be synchronized to the signal either by detecting peaks, or detecting a slope leading to a peak, and issuing therapy pulses in a closed loop manner.

Another approach is to determine the frequency of the missing characteristic frequency and simply deliver an output that is frequency matched to the missing or attenuated characteristic frequency, as indicated at 162. Square waves may be issued, or a more complex wave such as an actual sinusoidal wave or an emulated sinusoid, such as a digital approximation of a sinusoidal wave, may be generated.

The enhancing signal generated at 152 may be issued and the patient response monitored. Therapy may be preliminarily confirmed by monitoring for a change in the missing or attenuated characteristic frequency signal, as indicated at 164. Whether a change in the missing or attenuated characteristic frequency signal translates to an effect on patient symptoms may be determined as well, for example, using monitoring of any signals that were relied upon to identify the symptoms in the first place, as taught above relative to block 102 in FIG. 3A. If the missing or attenuated characteristic frequency and/or symptoms are not affected, further adjustments may be made, as indicated at 166. Adjustment 166 may include switching from one enhancement strategy to another, for example, or combining two such strategies, or changing a parameter of a strategy in use, such as increasing or decreasing one or more of amplitude, frequency, etc.

In some examples, the result may be considered an enhancement program that can be stored for later execution in the device. That is, if a change is observed in 164/166, and the missing or attenuated frequency appears to be enhanced, the therapy program can be stored to memory, and later called again in response to a trigger. To do so, the method may include block 170, which would trigger use of the enhancement signal by entering the method at block 152, as shown.

A trigger at block 170, prompting use of the stored enhancing signal, may be the same as at block 102 in FIG. 3A. A trigger may also include a determination that the Characteristic Frequency that was identified previously as missing, is no longer present in the sensed neural signals. That may be taken as indicating the possibility, likelihood, or occurrence in the near future, of symptom onset, and can be used as a trigger for therapy at block 170, if desired. Block 170 may also include storing one or more sensed signals preceding the initiation of therapy—for example, if sensing data is stored in a looping register (such as a first-in-first-out memory), the signals associated with or preceding the trigger can be stored for later physician review, if desired. Moreover, blocks 152 and/or 164 may also include recording one or more neural signals, to observe for example what occurs while the enhancing therapy is delivered at 152, or to observe neural activity after therapy delivery.

Turning briefly back to FIG. 3A, if, at block 106, there are multiple changes in frequency peaks, outputs in response may be as described above with respect to each of FIGS. 3B and 3C. For example, both a disruptive output and an enhancing output may be generated, within the same therapy program or as two separate therapy programs running concurrently and/or in an alternating pattern or sequence, as desired. If multiple extra frequency peaks have arisen, or if multiple frequency peaks are missing, two separate programs for therapy, one for each additional or missing frequency peak may be generated. Alternatively, a single therapy program attempting to address both additional or missing frequencies by disruption or enhancement in one program may be generated. Various combinations can be used, as desired.

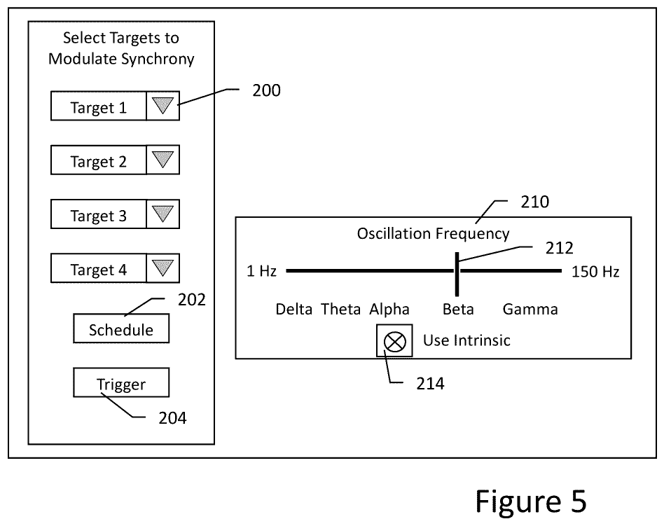

FIG. 4 was described previously, and so one turns to FIG. 5. Here, a graphical user interface, such as may appear in a clinician programmer (CP) is illustrated in simplified form. At 200, a plurality of available programs for issuing enhancing or disrupting therapy are available. These may be issued as part of a schedule as indicated at 202.

If desired, a trigger may be identified as indicated at 204. Triggers may include those described previously relative to FIGS. 3A-3C. In some examples, therapy can be triggered by the time of day or by a likely upcoming patient activity. For example, if the patient is waking up, or going to sleep, and the symptoms noted previously when performing FIGS. 3A-3C-type methods would interfere with the patient activity, an enhancing or disruptive signal may be generated as a preventive step, even in the absence of current symptoms. A trigger may include a task, such as a memory game or other patient activity that may result in a change in characteristic frequencies. In some examples, a cognitive task may be expected to enhance theta and/or gamma frequency bands, and stimulation may boost those same frequencies, and therefore improve performance. A therapy output may, for example, be prompted by the patient interacting with the RC to indicate a desired cognitive task or exercise is to be performed; in response, a stimulation output in the gamma frequency, or other pro-cognition band, may be generated. A physical task or exercise may instead be a trigger, for example, if a patient is prone to issues with falling, stability, or physical impairment (tremors for example), the implantable device may determine that the patient has changed posture, such as from sitting or lying to standing, and this may be trigger to engage therapy to prevent tremors in anticipation of physical exercise including movement, such as walking. Therapy programs can thus be configured to initiate in response to the pre-determined trigger conditions.

At 210, it can be seen that the nature of responsive therapy may be determined according to frequency. A slider at 212 may be used to select frequencies that are commonly known by convention for disruption or enhancement. In other examples, an intrinsic signal 214 can be used, either as the trigger or at the signal to be enhanced or disrupted.

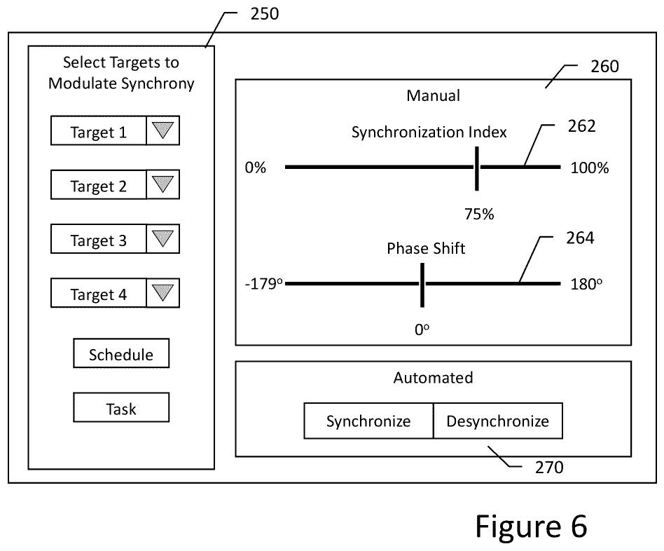

FIG. 6 shows another illustrative graphical user interface. At 250, another set of programs and a scheduler are illustrated, similar to FIG. 5. At 260, a manual control block is shown for configuring the nature of an output signal to be used. Here, the synchronization index can be set with slider 262, indicating how much additional synchronization is desirable in the particular patient. Phase shift for output therapy pulses can be set using slider 264. At 270, a toggle bar allows the user to determine whether the aim is to create additional synchrony or to desynchronize neural activity. Here, with block 270, an automated approach as in FIGS. 3A-3C may be used. Alternatively, with block 270, a shorter form approach may be to test and sense repeatedly, such as by monitoring for responsive changes to issued therapy as described in blocks 122, 136, and 138 in FIG. 3B (for disrupting synchrony), or at blocks 152, 164, and 166 of FIG. 3C (for enhancing synchrony).

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” Moreover, in the claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic or optical disks, magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description.

The Abstract is provided to comply with 37 C.F.R. § 1.72 (b), to allow the reader to quickly ascertain the nature of the technical disclosure. It is submitted with the understanding that it will not be used to interpret or limit the scope or meaning of the claims.

Also, in the above Detailed Description, various features may be grouped together to streamline the disclosure. This should not be interpreted as intending that an unclaimed disclosed feature is essential to any claim. Rather, innovative subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the protection should be determined with reference to the appended claims, along with the full scope of equivalents to which such claims are entitled.

What is claimed is:

1. A method of treating a patient having a neural condition with symptomatic episodes, the method comprising:
recording first neural activity while the patient is not experiencing a symptomatic episode;
receiving an indication of a symptomatic episode;
recording second neural activity during the symptomatic episode;
identifying characteristic frequencies of the first neural activity and characteristic frequencies of the second neural activity;
selecting a characteristic frequency occurring only during the second neural activity; and
issuing neural stimulation to disrupt neural activity at the selected characteristic frequency.

2. The method of claim 1, wherein the step of identifying characteristic frequencies includes performing a mathematical transformation of data for first neural activity and the second neural activity from a time domain to a frequency domain, and identifying peaks in the frequency domain for the first neural activity and the second neural activity.

3. The method of claim 1, wherein the step of issuing neural stimulation to disrupt the selected characteristic frequency includes identifying a source of the second neural activity having the selected characteristic frequency.

4. The method of claim 1, further comprising:
identifying a location of the second neural activity having the selected characteristic frequency; and
issuing neural stimulation to disrupt neural activity at the selected characteristic frequency by selecting electrodes for delivering the neural stimulation to deliver an electrical field at the location of the second neural activity.

5. The method of claim 1, wherein the step of issuing neural stimulation is performed using an implanted neural stimulator having a lead with a plurality of electrodes, the neural stimulator configured to selectively use subsets of the plurality of electrode to sense neural activity, and the method further comprises:
a) sensing neural activity using each of a first subset of the plurality of electrodes and a second subset of the plurality of electrodes;
b) determining which of the subsets of the plurality of electrodes used in a) attains a stronger signal at the selected characteristic frequency; and
c) issuing the neural stimulation using the subset of the plurality of electrodes identified in b).

6. The method of claim 1, wherein the step of issuing neural stimulation comprises:
sensing neural activity;
filtering the sensed neural activity to obtain signals at the selected characteristic frequency;
determining a time of peaks at the characteristic frequency; and
issuing the neural stimuli at a delay relative to the time of peaks.

7. The method of claim 6, wherein the delay is selected to achieve a phase delay in the range of 120 to about 240 degrees.

8. The method of claim 1, wherein the step of issuing neural stimulation is performed at a frequency having a period that is in the range of about 105% to 125% of a period of the characteristic frequency.

9. The method of claim 1, wherein the steps of recording first neural activity and second neural activity are performed with an implanted system.

10. The method of claim 1, wherein the step of issuing neural stimulation is performed in response to a trigger.

11. The method of claim 10, wherein the trigger is a patient input indicating occurrence of symptoms.

12. The method of claim 10, wherein the trigger is a detection of the characteristic frequency occurring only in the second neural activity.

13. A method of treating a patient having a neural condition with symptomatic episodes, the method comprising:
obtaining first neural activity representing an expected neural activity spectral data;
receiving an indication of a symptomatic episode;
recording second neural activity during the symptomatic episode;
identifying characteristic frequencies of the first neural activity and characteristic frequencies of the second neural activity;
selecting a characteristic frequency occurring only during the second neural activity; and
issuing neural stimulation to disrupt neural activity at the selected characteristic frequency.

14. The method of claim 13, wherein the step of identifying characteristic frequencies includes performing a mathematical transformation of data for the second neural activity from a time domain to a frequency domain, and identifying peaks in the frequency domain for the second neural activity.

15. The method of claim 13, wherein the step of issuing neural stimulation to disrupt the selected characteristic frequency includes identifying a source of the second neural activity having the selected characteristic frequency.

16. The method of claim 13, further comprising:
identifying a location of the second neural activity having the selected characteristic frequency; and
issuing neural stimulation to disrupt neural activity at the selected characteristic frequency by selecting electrodes for delivering the neural stimulation to deliver an electrical field at the location of the second neural activity.

17. The method of claim 13, wherein the step of issuing neural stimulation is performed using an implanted neural stimulator having a lead with a plurality of electrodes, the neural stimulator configured to selectively use subsets of the plurality of electrode to sense neural activity, and the method further comprises:
a) sensing neural activity using at least a first subset of the plurality of electrodes and a second subset of the plurality of electrodes;
b) determining which of the subsets of the plurality of electrodes used in a) attains a stronger signal at the selected characteristic frequency; and
c) issuing the neural stimulation using the subset of the plurality of electrodes identified in b).

18. The method of claim 13, wherein the step of issuing neural stimulation comprises:
sensing neural activity;
filtering the sensed neural activity to obtain signals at the selected characteristic frequency;
determining a time of peaks of the neural activity in the characteristic frequency; and
issuing the neural stimuli at a delay relative to the time of peaks.

19. A method of treating a patient having a neural condition with symptomatic episodes, the method comprising:
recording first neural activity while the patient is not experiencing a symptomatic episode;
receiving an indication from a patient of a symptomatic episode;
recording second neural activity during the symptomatic episode;
identifying characteristic frequencies of the first neural activity and characteristic frequencies of the second neural activity;
selecting a characteristic frequency occurring only in the first neural activity; and
issuing neural stimulation to enhance neural activity at the selected characteristic frequency.

20. The method of claim 19, wherein the step of identifying characteristic frequencies includes performing a mathematical transformation of data for the first neural activity and the second neural activity from a time domain to a frequency domain, and identifying peaks in the frequency domain for the first neural activity and the second neural activity.
